(19) United States
(12) Patent Application Publication
(10) Pub. No.: US 2009/0131977 A1
(43) Pub. Date: May 21, 2009

(54) COMBINATION TWEEZER AND EYE HAIR GROOMING DEVICE AND METHOD

(76) Inventor: Richard Ross, Henderson, NV (US)

Correspondence Address: WEISS & MOY PC, 4204 NORTH BROWN AVENUE, SCOTTSDALE, AZ, 85251, US

(21) Appl. No.: 11/943,840

(22) Filed: Nov. 21, 2007

(51) Int. Cl. A61B 17/50; B25B 9/00

(52) U.S. Cl. 606/211; 294/99.2

(57) ABSTRACT

A combination tweezer and eye hair care device and method. A combination device includes a tweezer along a first portion of the length thereof and an eye hair care device along a second portion. The eye hair care device may be an eyebrow brush or an eyelash comb In one embodiment, the eye hair care device is removable. The tweezer may have a straight, angled or pointed cutting tip.

### FIELD OF THE INVENTION

The present invention relates generally to personal grooming devices and, more particularly, to a combination tweezer and eye hair grooming device and method.

### BACKGROUND OF THE INVENTION

Women may use a variety of personal grooming devices related to hair surrounding the eyes. For example, a tweezer may be used to pluck unwanted eyebrow hair. An eyebrow brush may be used to brush eyebrow hair. And, finally, an eyelash comb may be used to comb the eyelashes. Such tools are provided individually, and it may be inconvenient for a person, particularly when in a hurry, to locate multiple such tools in a crowded make-up case or purse.

The present invention is directed to a combination tweezer and eye hair grooming device and method, which in one embodiment may be an eyelash comb and, in another, an eyebrow brush.

### SUMMARY OF THE INVENTION

In accordance with an embodiment of the present invention, a combination tweezer and eye hair device is disclosed. The device comprises, in combination: a tweezer, occupying a first portion of a length of a combination device terminating at a first end of the combination device in a cutting tip; and an eye hair care device occupying a second portion of the length of the combination device and positioned proximate a second end of the combination device.

In accordance with another embodiment of the present invention, a combination tweezer and eye hair care device is disclosed. The device comprises, in combination: a tweezer, occupying a first portion of a length of a combination device terminating at a first end of the combination device in a cutting tip; and an eye hair care device occupying a second portion of the length of the combination device and positioned proximate a second end of the combination device; wherein the eye hair care device is removable from the combination device.

In accordance with a further embodiment of the present invention, a care method for eye hair is disclosed. The method comprises: providing a tweezer, occupying a first portion of a length of a combination device terminating at a first end of the combination device in a cutting tip; providing an eye hair care device occupying a second portion of the length of the combination device and positioned proximate a second end of the combination device; a user utilizing the tweezer to remove at least one eye hair; and a user inverting the combination device and utilizing the eye hair care device to care for eye hair.

### BRIEF DESCRIPTION OF THE DRAWINGS

FIG. 1 is a side view of a combination tweezer and eye hair tool consistent with an embodiment of the present invention.

FIG. 2 is a side view of a combination tweezer and eye hair tool consistent with another embodiment of the present invention.

FIG. 3 is a side view of a combination tweezer and eye hair tool consistent with a further embodiment of the present invention.

FIG. 4 is a bottom view of a combination tweezer and eye hair tool consistent with an embodiment of the present invention.

FIG. 5 is a top view of a combination tweezer and eye hair tool consistent with an embodiment of the present invention.

FIG. 6 is a side view of a portion of a combination tweezer and eye hair tool consistent with an embodiment of the present invention.

FIG. 7 is a side, exploded view of the combination tweezer and eye hair tool of FIG. 6.

FIG. 8 is a side view of a portion of a combination tweezer and eye hair tool consistent with another embodiment of the present invention.

FIG. 9 is a side view of a portion of a combination tweezer and eye hair tool consistent with a further embodiment of the present invention.

### DETAILED DESCRIPTION OF THE PREFERRED EMBODIMENTS

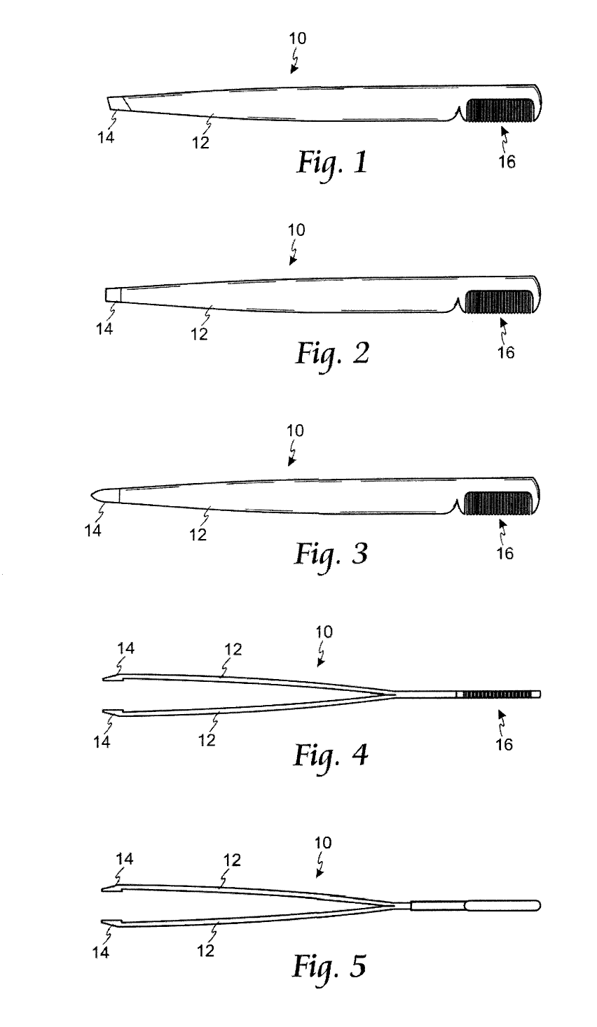

Referring first to FIGS. 1-5, embodiments of a combination tweezer and eye hair device 10 (hereinafter “device 10”) are illustrated. The device 10 includes a pair of tweezer arms 12, terminating in a tweezer cutting tip 14. The device 10 further comprises an eyelash comb 16, positioned proximate an end thereof, opposite the tweezer cutting tip 14. In this regard, it can be seen that, in this embodiment, the device 10 is a combination tweezer and eyelash comb in a single tool.

Referring more specifically to FIGS. 1-3, it can be seen that, in one embodiment, the cutting tip 14 is angled (see FIG. 1), in another it may have a straight edge (see FIG. 2) and that it may instead be provided in a pointed configuration (see FIG. 3).

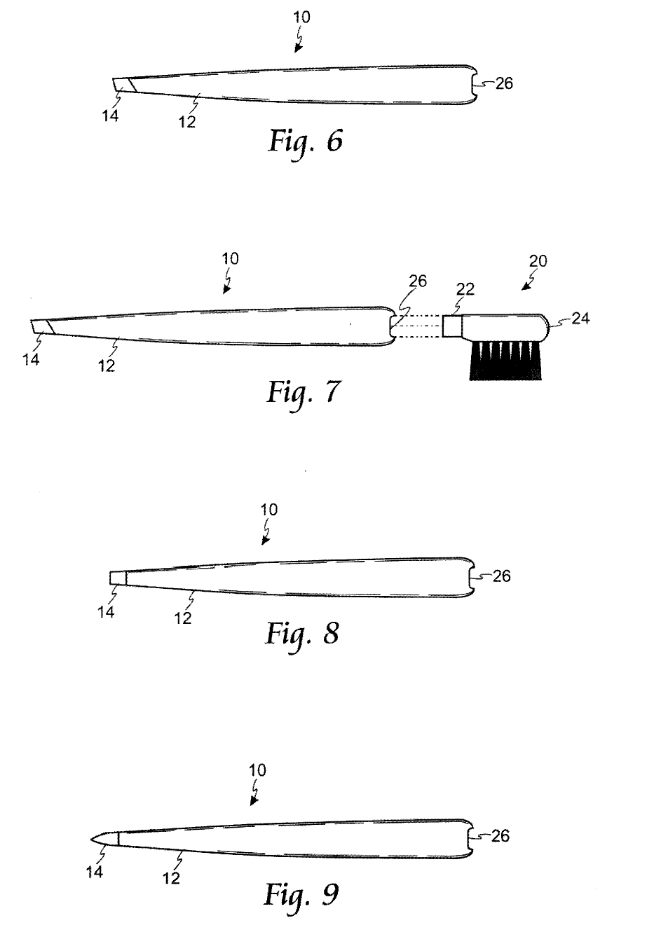

Referring now to FIGS. 6-9, another embodiment of a combination tweezer and eye hair device 10 is illustrated. The device 10 includes a pair of tweezer arms 12, terminating in a tweezer cutting tip 14. The device 10 further comprises an eyebrow brush 20, positioned proximate an end thereof, opposite the tweezer cutting tip 14. In this regard, it can be seen that, in this embodiment, the device 10 is a combination tweezer and eyelash comb in a single tool.

As best seen in FIG. 7, it can be seen that the brush 20 may be provided as a removable attachment to the device 10, wherein an end portion 22 of a base portion 24 of the brush 20 nay be inserted into a corresponding opening 26 in the device 10. Such a configuration may permit a user to remove the brush 20 when not needed, to replace it when worn, to utilize brushes 20 of varying sizes with a single device 10. It should be noted that while a removable brush 20 is illustrated, it may be desired to provide a device 10 having a removable eyelash comb (not shown) instead.

As noted above with respect to FIGS. 1-3, it can be seen by reference to FIGS. 7-9 that the cutting tip 14 of the tweezer portion of the device 10 of this embodiment may be angled (see FIG. 7), in another it may have a straight edge (see FIG. 8) and that it may instead be provided in a pointed configuration (see FIG. 9).

While the invention has been particularly shown and described with reference to preferred embodiments thereof, it will be understood by those skilled in the art that the foregoing and other changes in form and details may be made therein without departing from the spirit and scope of the invention.

I claim:

1. A combination tweezer and eye hair device comprising, in combination:
a tweezer, occupying a first portion of a length of a combination device terminating at a first end of the combination device in a cutting tip; and
an eye hair care device occupying a second portion of the length of the combination device and positioned proximate a second end of the combination device.

2. The device of claim 1 wherein the eye hair care device is an eyebrow brush.

3. The device of claim 1 wherein the eye hair care device is an eyelash comb.

4. The device of claim 1 wherein the cutting tip has a straight edge.

5. The device of claim 1 wherein the cutting tip has an angled edge.

6. The device of claim 1 wherein the cutting tip is pointed.

7. A combination tweezers and eye hair device comprising, in combination:
a tweezer, occupying a first portion of a length of a combination device terminating at a first end of the combination device in a cutting tip; and
an eye hair care device occupying a second portion of the length of the combination device and positioned proximate a second end of the combination device;
wherein the eye hair care device is removable from the combination device.

8. The device of claim 7 wherein the eye hair care device is an eyebrow brush.

9. The device of claim 7 wherein the eye hair care device is an eyelash comb.

10. The device of claim 7 wherein the cutting tip has a straight edge.

11. The device of claim 7 wherein the cutting tip has an angled edge.

12. The device of claim 7 wherein the cutting tip is pointed.

13. A care method for eye hair comprising:
providing a tweezer, occupying a first portion of a length of a combination device terminating at a first end of the combination device in a cutting tip;
providing an eye hair care device occupying a second portion of the length of the combination device and positioned proximate a second end of the combination device;
a user utilizing the tweezers to remove at least one eye hair; and
a user inverting the combination device and utilizing the eye hair care device to care for eye hair.

14. The method of claim 13 wherein the eye hair care device is an eyebrow brush.

15. The method of claim 13 wherein the eye hair care device is an eyelash comb.

16. The method of claim 13 wherein the cutting tip has a straight edge.

17. The method of claim 13 wherein the cutting tip has an angled edge.

18. The method of claim 13 wherein the cutting tip is pointed.

19. The method of claim 13 wherein the eye hair care device is removable from the combination device.
